(19) United States
(12) Patent Application Publication
(10) Pub. No.: US 2012/0016270 A1
(43) Pub. Date: Jan. 19, 2012

(54) FALL DETECTION DEVICE

(75) Inventors: Stephan Bühler, Zug (CH); Patrick Favre, Bussigny-Pres-Lausanne (CH)

(73) Assignee: SYNAPSE S.A.R.L., Bienne (CH)

(21) Appl. No.: 13/257,477

(22) PCT Filed: Mar. 16, 2010
(86) PCT No.: PCT/CH2010/000071

§ 371 Date: Oct. 5, 2011

(30) Foreign Application Priority Data
Mar. 20, 2009 (CH) 428/09

(51) Int. Cl. A61B 5/11

(52) U.S. Cl. 600/595

(57) ABSTRACT

A fall detection device (10) which comprises two identical housings (11), each containing an identical pressure sensor (12) housed within a suitable recess (13) of the corresponding housing (11). The housings (11) are mounted on a printed circuit (14) using electrical connection pins (15). The two sensors (12) are arranged coaxially head to foot and communicate directly with the ambient air in order to output a measurement of the atmospheric pressure value. The sensors are arranged so as to generate a signal based on the measurements utilized for detecting a fall, and comprise a unit for transmitting the signal to a transmission station as an alarm signal.

This application is a National Stage completion of PCT/CH2010/000071 filed Mar. 16, 2010, which claims priority from Swiss patent application serial no. 428/09 filed Mar. 20, 2009.

### FIELD OF THE INVENTION

The present invention concerns a fall detection device especially for detecting when a person has fallen, in order to transmit a warning signal and generate a call for help if necessary, comprising at least one housing in which at least two atmospheric pressure sensors are mounted.

### PRIOR ART TECHNIQUE

Numerous devices exist to automatically detect a fall by an elderly person living alone or an isolated worker, for example, and to engage an alarm and initiate a call for help if necessary.

Several devices that fulfill this function are available on the market. Detection occurs through the use of speed detectors, three dimensional accelerometers or position detectors that identify the fact that a person has moved from a vertical to an inclined or horizontal position, and which emit a signal following detection. These devices have a high rate of error in the form of what is called positive errors, that is, they engage a signal on the basis of an erroneous interpretation; for example, if the wearer moves suddenly when playing cards, awakens abruptly or stoops to retrieve an object; or in the form of negative errors, that is, they do not engage a signal when the wearer falls slowly, such as when holding onto a wall or leaning against an object.

Such an apparatus is described, for example, in European Patent Publication No. 1 731 098 A1, the object of which is a “Method for Detecting Fall by a Person” using at least one accelerometer and a magnetomer. A fall is identified when the apparatus registers a significant and rapid oscillation of the acceleration signal that coincides with a variation in the ambient magnetic field.

Another detection device for detecting, signaling and transmitting data is disclosed in European Patent Application published as No. 1 632 920 A1, which describes a portable device comprising at least one shock sensor and a fall sensor integrating a miniature internal unit comprising accelerometers, gyroscopes and a means for transmitting an alarm signal.

Another automatic fall detection device is described in International Publication WO 2004/100092 A2, a device designed to measure acceleration, detect acceleration, and transmit a signal to a remote receptor that engages an alarm to call for help.

European Patent Application Publication No. 1 870 037 A1 describes an apparatus to detect when the user has fallen and become immobilized, which comprises at least one sensor that furnishes, if necessary, an acceleration signal and a signal indicating the user's position. U.S. Pat. No. 3,667,293 describes a bimorphous sensor designed to measure pressure variations, specifically for measuring altitude in aeronautics. This type of sensor does not operate on the same basis as the device of the invention and it has an unacceptable rate of error in fall detection. In practice it uses either a single sensor or two sensors subjected on one side to air pressure and on the other to a vacuum. In the device according to the present invention, to correctly identify a fall, both sensors used are always arranged coaxially head to foot and the ambient air is directed equally over each of them, thus separating the effects of gravity and greatly improving reliability of measurement.

### DESCRIPTION OF THE INVENTION

The innovative solution proposed provides a certain and reliable answer to the problem of identifying a fall by a person wearing the device of the invention through the use of a simple, economical and miniaturized means that can be integrated into various objects a user might wear without embarrassment or esthetic concern.

To achieve this, the fall detection device of the invention as described in The preamble is characterized in that the sensors are designed so they both communicate directly with the ambient air to furnish an absolute measurement of the atmospheric pressure value, and in that they are arranged coaxially head to foot in order to nullify the effects of gravity on the difference in atmospheric pressure generated during a fall.

According to a preferred embodiment of the invention, the detectors are preferably identical and are absolute pressure type detectors designed to deliver an absolute measurement of the atmospheric pressure value.

In this embodiment, the at least one housing comprises two cavities which house the two detectors, respectively, each of the cavities being equipped with a least one opening to admit ambient air equally over the two atmospheric pressure detectors.

Advantageously the device comprises a means for generating the alarm signal as a function of the atmospheric pressure measurement values performed by the sensors and a means for transmitting this alarm signal to a transmitting station.

According to a second preferred embodiment, the device comprises two pairs of atmospheric pressure sensors designed so that both communicate directly with the ambient air and arranged so that the sensors in each of the pairs are disposed coaxially head to foot, the sensors in one of the pairs being mounted along a first axis and the sensors in the other pair being mounted along a second axis generally perpendicular to the first axis.

The sensors in each pair are advantageously identical and deliver an absolute measurement of the atmospheric pressure value.

According to a third preferred embodiment, the device may comprise three pairs of atmospheric pressure sensors, designed so that all three communicate directly with the ambient air and arranged in such a way that the sensors in each of the pairs are arranged coaxially head to foot and mounted along three axes that are respectively perpendicular to one another.

### SUMMARY DESCRIPTION OF THE DRAWINGS

The present invention and its features will be more readily apparent from the following description of one embodiment given by way of non-limiting example, with reference to the attached drawings in which:

FIG. 1 represents a perspective view of a first embodiment of the device according to the invention;

FIG. 1A is a lateral elevational view of the device of FIG. 1 taken along the direction defined by arrow A;

FIG. 1B is an overhead view of the device of FIG. 1 taken along the direction of arrow B;

FIG. 1C is a cross-section along a plane section integrating axis C-C in the device of FIG. 1;

FIG. 2 is a perspective view of an embodiment that is a variation of the device of FIG. 1;

FIG. 2A is an elevational view taken along the direction of arrow A in FIG. 2;

FIG. 2B is an overhead view taken along the direction of arrow B in FIG. 2;

FIG. 2C is a cross-section along a plane section integrating axis C-C of the device of FIG. 2;

FIG. 2D is a cross section along a plane section integrating axis D-D of the device of FIG. 2;

FIG. 3 is a perspective view of a second form of embodiment of the device according to the invention;

FIG. 3A is an elevational view taken along the direction of arrow A in FIG. 3;

FIG. 3B is an overhead view of the device of FIG. 3;

FIG. 3C is a cross-section along a plane section integrating axis C-C of the device in FIG. 3;

FIG. 3D represents a cross-section along a plane section integrating axis D-D of the device in FIG. 3;

FIG. 4 represents a perspective view of a third form of embodiment of the device according to the invention;

FIG. 4A is an elevational view taken along the direction of arrow A in FIG. 4;

FIG. 4B is an overhead view taken along the direction of arrow B in FIG. 4;

FIG. 4C is a cross-section along a plane section integrating axis C-C in the device of FIG. 4; and

FIG. 4D is a cross-section along a plane section integrating axis D-D of the device in FIG. 4.

### DETAILED DESCRIPTION OF THE PREFERRED EMBODIMENTS

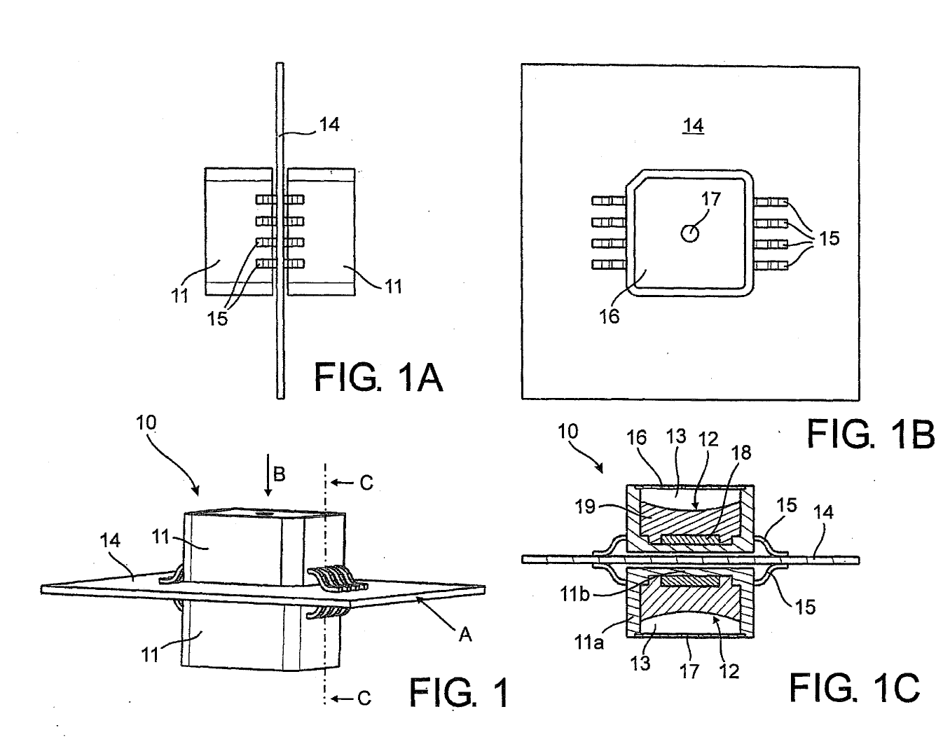

With reference to FIGS. 1, 1A, 1B and 1C, a fall detection device 10 is composed of two identical housings 11 each containing an identical sensor 12 housed in an appropriate cavity 13 in the corresponding housing 11. The sensors are pressure type sensors that can be made of different components, for example, piezo-electric sensors, quartz sensors, or silicon sensors. Housings 11 are mounted on a printed circuit 14 by means of electrical connecting pins 15.

The cavities 13 in which sensors 12 are housed are blocked by a closing plate 16 constituting a sort of cover abutting lateral walls 11a of housing 11, the closing plate 16 being equipped with an opening 17 allowing cavity 13 containing sensor 12 to communicate with the ambient air. Sensor 12 takes the form of an electronic chip 18 disposed on base 11b of housing 11 and connected to electrical connecting pins 15. This chip 18 is covered with a layer of gel seal 19 to protect it from humidity.

The two sensors 12 are preferably identical but arranged coaxially head to foot. This arrangement serves to nullify the effects of gravity on the difference in atmospheric pressure and as a result, the difference in height, thereby allowing extremely precise measurements to be taken. The pressure difference acts simultaneously on the two sensors. Gravity or other forces external to the fall may be separated from the fall itself. The device of FIG. 1 performs measurements in a single direction, which follows the axis of the sensors, shown by arrow B, the axis being essentially perpendicular to substrate 14 of the printed circuit. Openings 17 are designed to allow ambient air to flow equally over both sensors 12. Analysis of the measurements obtained by the two sensors eliminates any disturbances caused by airflow. Once the measurement of atmospheric pressure is obtained through the measurements performed by the pair of sensors that function simultaneously and are positioned head to foot, it is possible to determine the force acting upon the sensors. This information, combined with the time, permits acceleration to be calculated and defines the speed of the fall.

Electronic chip 18 advantageously comprises a means of generating a signal as a function of the fall detection measurements and a means of transmitting this signal, preferably automatically as an alarm signal, to a transmitting station designed to react by summoning help using an appropriate structure.

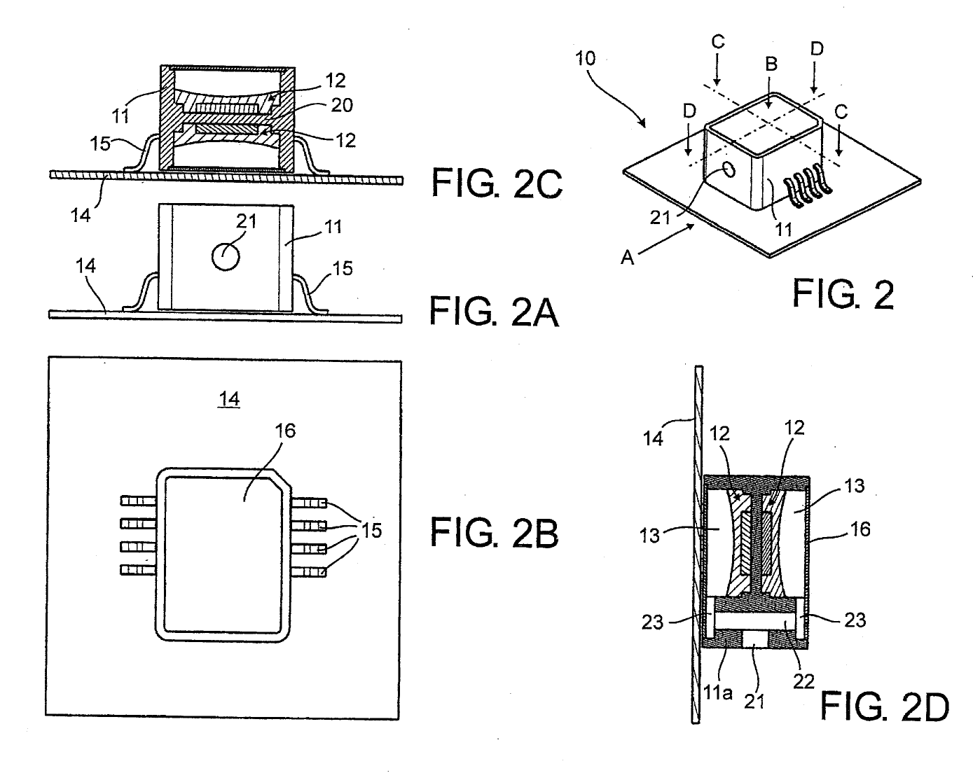

With reference to FIGS. 2, 2A, 2B, 2C, and 2D, the device 10 is composed of a single housing 11 containing two identical sensors 12 respectively housed in two suitable cavities 13 in housing 11. The housing 11 is mounted on a printed circuit 14 by means of electrical connection pins 15. Cavities 13 are blocked by a closing plate 16 constituting a sort of cover abutting lateral walls 11a of housing 11. The two cavities 13 are separated by a central wall 20 dividing the housing into two identical compartments. In this case closing plates 16 do not have openings for the passage of atmospheric air. Air passes through an opening 21 penetrating a lateral wall 11a in housing 11 that opens onto a lateral passageway 22 communicating with two chimneys 23 connecting the passageway 22 with the respective cavities 13 in the two receptors 12.

The operating principle and features of this device are essentially similar to those of FIG. 1. The sensors are disposed on the same side of the printed circuit, which may offer design advantages, depending upon the application desired and on the implantation of the sensors into devices such as watches, pendants, bracelets, or other utilitarian objects.

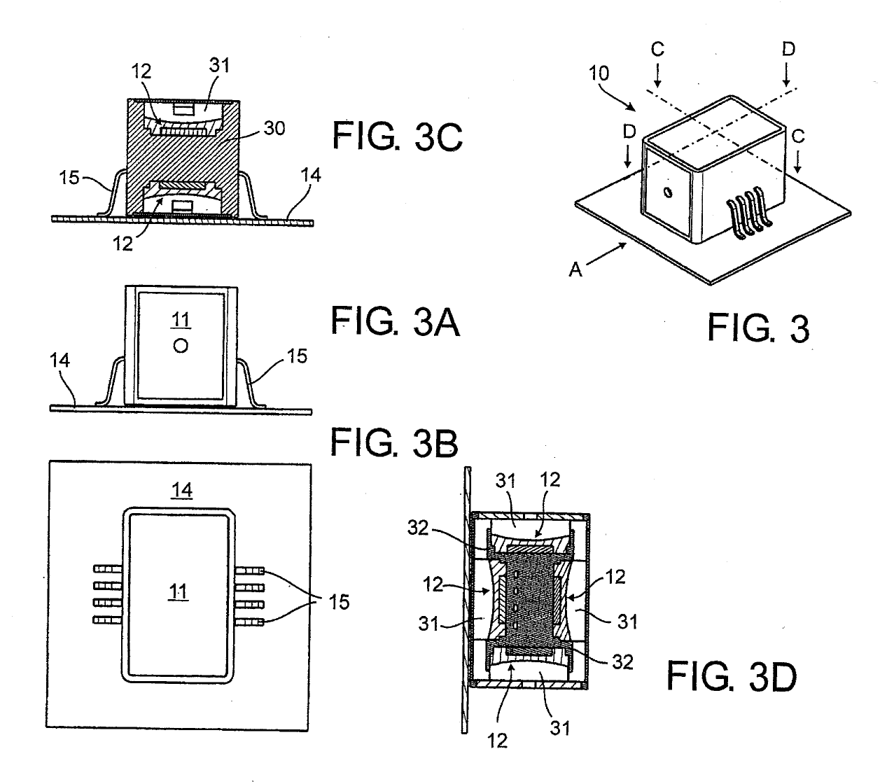

FIGS. 3, 3A, 3B, 3C and 3D illustrate a second embodiment of device 10 in which housing 11 contains four identical sensors 12. As described previously, housing 11 is mounted on a printed circuit 14 using electrical connection pins 15. Housing 11 comprises a central block 30 that serves as a support for two pairs of sensors 12 disposed two by two, head to foot, along two perpendicular axes. This central support defines four cavities 31 with its arms 32, in which the sensors are located. As before, cavities 31 are directly open to the ambient air either because of the openings that exist in the closing plates of the cavities, or because of indirect passageways that place the cavities in communication with the ambient air.

This embodiment performs measurements along the two orthogonal axes and consequently identifies a vertical fall or an oblique fall and determines the directional components of the fall according to the two perpendicular axes.

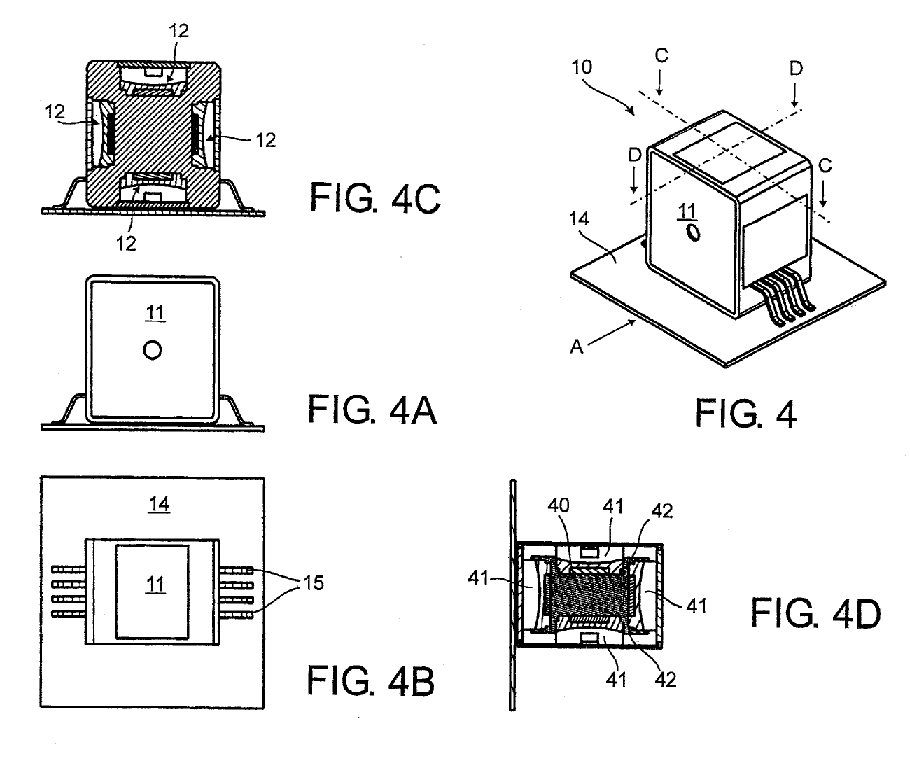

In a third embodiment shown in FIGS. 4, 4A, 4B, 4C and 4D, device 10 is composed of a single housing 11 containing six identical sensors 12 mounted on a printed circuit 14 using electrical connection pins 15.

Housing 11 comprises a central block 40 that serves as a support for three pairs of sensors 12 disposed two by two and head to foot along three perpendicular axes. This central support 40 defines six cavities 41 by arms 42 which create housings where the sensors are located. As before, cavities 41 are directly open to the ambient air either because of the openings that exist in the closing plates of the cavities, or because of indirect passageways that place the cavities in communication with the ambient air.

This embodiment performs measurements along three orthogonal axes and consequently identifies a vertical fall or an oblique fall and determines the directional components of the fall according to the three perpendicular axes.

The present invention resolves the problem posed, that is, the problem of fall detection. When a fall is effectively detected, the signal generated may be amplified by cascading amplifiers integrated into the housings to reduce their bulk, which have low parasitic emission due to the use of a high precision analog-digital converter with a low parasitic level. The signal is then communicated by a wire connection or by wireless wave transmission to a monitoring service that manages possible emergency intervention. The device has conventional applications in the sector of caring for the sick or elderly, but also in the arena of sports, such as parachuting, kite surfing, mountain sports, etc., or for protecting workers in an isolated, potentially dangerous environment.

1-8. (canceled)

9. A fall detection device (10), for detecting when a person has fallen, in order to transmit a warning signal and generate a call for help if necessary, the device (10) comprising:
at least one housing (11) in which at least two atmospheric pressure sensors are mounted, the sensors (12) being designed both to communicate directly with ambient air in order to furnish a measurement of atmospheric pressure values, and the sensors (12) being arranged coaxially head to foot in order to nullify effects of gravity on a difference in atmospheric pressure generated by a fall.

10. The device according to claim 9, wherein the sensors (12) are identical and are absolute pressure type sensors, and the sensors (12) furnish an absolute measurement of the atmospheric pressure value.

11. The device according to claim 9, wherein the at least one housing (11) comprises two cavities (13) designed to house the two sensors (12) respectively, each of the cavities is equipped with at least one opening (17) which allows ambient air to flow equally over the two atmospheric pressure sensors (12).

12. The device according to claim 9, wherein the device (10) comprises a means for generating the warning signal as a function of the atmospheric pressure measurement values taken by the sensors (12) and a means for transmitting the warning signal to a transmitting station.

13. The device according to claim 9, wherein the device (10) comprises two pairs of atmospheric pressure sensors (12) which are designed such that both pairs of atmospheric pressure sensors (12) communicate directly with the ambient air and the pairs of atmospheric pressure sensors (12) are disposed such that the sensors, in each of the pairs of atmospheric pressure sensors (12), are arranged coaxially head to foot.

14. The device according to claim 13, wherein the sensors (12) in one of the pairs of atmospheric pressure sensors (12) are mounted along a first axis, while the sensors in the other pair of atmospheric pressure sensors (12) are mounted along a second axis which is substantially perpendicular to the first axis.

15. The device according to claim 14, wherein the sensors (12) in each of the pairs of atmospheric pressure sensors (12) are identical and furnish an absolute measurement of the atmospheric pressure value.

16. The device according to claim 10, wherein the device (10) comprises two pairs of atmospheric pressure sensors (12) which are designed such that both pairs of atmospheric pressure sensors (12) communicate directly with the ambient air and the pairs of atmospheric pressure sensors (12) are disposed such that the sensors, in each of the pairs of atmospheric pressure sensors (12), are arranged coaxially head to foot.

17. The device according to claim 16, wherein the sensors (12) in one of the pairs of atmospheric pressure sensors (12) are mounted along a first axis, while the sensors in the other pair of atmospheric pressure sensors (12) are mounted along a second axis which is substantially perpendicular to the first axis.

18. The device according to claim 17, wherein the sensors (12) in each of the pairs of atmospheric pressure sensors (12) are identical and furnish an absolute measurement of the atmospheric pressure value.

19. The device according to claim 9, wherein the device (10) comprises three pairs of atmospheric pressure sensors (12), all three of the pairs of atmospheric pressure sensors (12) are designed to communicate directly with the ambient air and disposed in such a way that the sensors, in each of the pairs of atmospheric pressure sensors (12), are arranged coaxially head to foot and mounted along three axes respectively perpendicular to one another.

20. The device according to claim 10, wherein the device (10) comprises three pairs of atmospheric pressure sensors (12), all three of the pairs of atmospheric pressure sensors (12) are designed to communicate directly with the ambient air and disposed in such a way that the sensors, in each of the pairs of atmospheric pressure sensors (12), are arranged coaxially head to foot and mounted along three axes respectively perpendicular to one another.

21. A fall detection device (10) for detecting when a person has fallen, and transmitting a warning signal and generating a call for help when the device (10) detects that the person has fallen, the device (10) comprising:
at least two atmospheric pressure sensors (12) being mounted within at least one housing (11), the at least two atmospheric pressure sensors (12) communicate directly with ambient air and measure atmospheric pressure values; and
the at least two atmospheric pressure sensors (12) are mounted coaxially head to foot for nullifying effects of gravity on differences of atmospheric pressure values being generated by the fall of the person.
